(19) United States
(12) Patent Application Publication
(10) Pub. No.: US 2020/0342963 A1
(43) Pub. Date: Oct. 29, 2020

(54) METHODS AND SYSTEMS FOR TELEHEALTH CONSULTATION

(71) Applicant: American Well Corporation, Boston, MA (US)

(72) Inventor: Ash MOHAMMAD, Woodland Hills, CA (US)

(21) Appl. No.: 16/397,850

(22) Filed: Apr. 29, 2019

(51) Int. Cl. G16H 10/60; G06Q 20/14; G16H 15/00

(52) U.S. Cl. CPC G16H 10/60; G16H 15/00; G06Q 20/14

(57) ABSTRACT

Embodiments provide a method or a system for integrating multiple processed involved in telehealth consultation. The method includes connecting the patient with the telehealth providers while monitoring status of the telehealth providers to provide quick consultation to the patients. The method integrates process of bill generation and medical report generation and other procedurals with the consultation process. The medical report and bills are generated as soon as the consultation is over based on medical inputs and billing details provided by the telehealth providers.

### TECHNICAL FIELD

The present disclosure relates to telehealth consultation and, more particularly to, methods and systems for integrating multiple processes which take place during telehealth consultation.

### BACKGROUND

Telehealth consultation is a process of evaluation and treatment of a medical condition of a patient by a doctor over distance through text, audio or video media. Telehealth consultation involves usage of information technology and telecommunication for providing access to health assessments, diagnoses, interventions, supervision, consultations, education, and information across a distance. In conventional medical treatment methods, sometimes patient visiting a nearby hospital for consulting a doctor regarding a health condition may face a situation that the required doctor is not available in the nearby hospital. In such a scenario, the patient has to go and visit another hospital which may be very far from the patient's location. Visiting a faraway hospital is inconvenient and in case of some emergency, delay in assistance or consultation may be very risky. Therefore, the telehealth consultation is an increasingly used initiative which makes the medical assistance reachable to everyone with no hassle of visiting multiple hospitals or faraway hospitals.

The present telehealth consultation systems are able to provide health consultation over a distance, however a number of processes such as billing the consultation or managing multiple telehealth providers (interchangeable referred as telehealth doctors) associated with the telehealth consultation system, keeping quality check of each consultation, maintaining salary of each staff members of the telehealth consultation system are still scattered processes and require a lot of manual intervention leading to errors in medical documentation of the patient or delays in the consultation process. Therefore there is a need for an improved telehealth consultation system which overcomes the above-stated problems.

### SUMMARY

Various embodiments of the present disclosure provide methods and systems for checking network connectivity before performing a payment transaction.

In an embodiment, an integrated telehealth management method is disclosed. The method includes receiving a consultation request for health consultation of a patient from a medical facility. The consultation request corresponds to an emergency. The consultation request comprising personal and health details of the patient. The method further includes sending the consultation request for health consultation of the patient to a plurality of telehealth providers. The method further includes sending a notification to the plurality of telehealth providers indicating the assigned consultation request. The method further includes receiving a reply from one or more telehealth providers of the plurality of telehealth providers. The reply comprising an availability status of the one or more telehealth providers and a waiting time of the one or more telehealth providers required before starting a session for the health consultation of the patient. The method further includes selecting a telehealth provider from the one or more telehealth providers based on the availability status of the telehealth provider and a least waiting time among the plurality of telehealth providers. The method further includes assigning the consultation request to the selected telehealth provider. The method further includes sending a notification to the medical facility indicating the assigned telehealth provider and an associated waiting time of the telehealth provider. The method further includes tracking in real-time an on-going health check-up session of the assigned telehealth provider. The method further includes determining completion of the on-going session of the assigned telehealth provider. Upon determination, the method further includes initiating a health check-up session of the patient with the assigned telehealth provider.

In another embodiment, an integrated telehealth management server system is disclosed. The server system includes a memory configured to store instructions and at least one processor configured to execute the stored instructions to cause the server system to perform the method. The method includes receiving, by the server system, a consultation request for health consultation of a patient from a medical facility. The consultation request corresponds to an emergency. The consultation request comprising personal and health details of the patient. The method further includes sending, by the server system, the consultation request for health consultation of the patient to a plurality of telehealth providers. The method further includes sending, by the server system, a notification to the plurality of telehealth providers indicating the assigned consultation request. The method further includes receiving, by the server system, a reply from one or more telehealth providers of the plurality of telehealth providers. The reply comprising an availability status of the one or more telehealth providers and a waiting time of the one or more telehealth providers required before starting a session for the health consultation of the patient. The method further includes selecting, by the server system, a telehealth provider from the one or more telehealth providers based on the availability status of the telehealth provider and a least waiting time among the plurality of telehealth providers. The method further includes assigning, by the server system, the consultation request to the selected telehealth provider. The method further includes sending, by the server system, a notification to the medical facility indicating the assigned telehealth provider and an associated waiting time of the telehealth provider. The method further includes tracking, by the server system, in real-time an on-going health check-up session of the assigned telehealth provider. The method further includes determining, by the server system, completion of the on-going session of the assigned telehealth provider. Upon determination, the method further includes initiating, by the server system, a health check-up session of the patient with the assigned telehealth provider.

In yet another embodiment, an integrated telehealth management method is disclosed. The method includes receiving a consultation request for health consultation of a patient from a medical facility. The consultation request comprising personal and health details of the patient, and a type of the consultation request. The type of the consultation request is one of an emergency or a regular health-checkup. The method further includes determining whether the type of the consultation request is the emergency or the regular health-checkup. Upon determination that the type of the consultation request is the regular health-checkup, the method further includes (1) assigning the consultation request for health consultation of the patient to a primary telehealth provider; (2) sending a notification to the primary telehealth provider indicating the assigned consultation request for health consultation of the patient; (3) receiving a reply from the primary telehealth provider, the reply comprises one of a video call, a phone call or a message from the primary telehealth provider. The reply indicating at least one of an availability status of the primary telehealth provider or a waiting time required before starting a session for the consultation request; (4) assigning the consultation request to the primary telehealth provider; (5) sending a notification to the medical facility indicating the associated waiting time for the primary telehealth provider; (6) tracking in real-time an on-going health check-up session of the primary telehealth provider; (7) determining completion of the on-going session of the primary telehealth provider; and (8) upon determination, initiating a health check-up session of the patient with the primary telehealth provider. The method further includes upon determination that the type of the consultation request is the emergency: (1) sending the consultation request for health consultation of the patient to a plurality of telehealth providers; (2) sending a notification to the plurality of telehealth providers indicating the assigned consultation request for health consultation of the patient; (3) receiving a reply from one or more telehealth providers of the plurality of telehealth providers, the reply comprising an availability status of the one or more telehealth providers and a waiting time of the one or more telehealth providers required before starting a session for the health consultation of the patient; (4) selecting a telehealth provider from the one or more telehealth providers based on the availability status of the telehealth provider and a least waiting time among the plurality of telehealth providers; (5) assigning the consultation request for health consultation of the patient to the selected telehealth provider; (6) sending a notification to the medical facility indicating the associated waiting time for the selected telehealth provider; (7) tracking in real-time an on-going health check-up session of the selected telehealth provider; (8) determining completion of the on-going session of the selected telehealth provider; (9) upon determination, initiating a health check-up session of the patient with the selected telehealth provider.

Other aspects and example embodiments are provided in the drawings and the detailed description that follows.

### BRIEF DESCRIPTION OF THE FIGURES

For a more complete understanding of example embodiments of the present technology, reference is now made to the following descriptions taken in connection with the accompanying drawings in which:

FIG. 1 illustrates an example representation of an environment 100, in which at least some example embodiments of the present disclosure can be implemented;

FIG. 2 illustrates a simplified block diagram of a server system used for integrating multiple processes involved in telehealth consultation procedure, in accordance with an example embodiment;

FIG. 3 illustrates a work station example of a telehealth provider facility including different departments, in accordance with an example embodiment;

FIG. 4 illustrates a flow diagram of a method for integrating multiple processes involved in telehealth consultation procedure, in accordance with an example embodiment;

FIG. 5 illustrates an example representation of a UI displayed to a display screen the computing devices of different employees or users of the telehealth provider system, in accordance with an example embodiment;

FIG. 6 illustrates an example representation of another UI displayed to the staff member of the call center department to facilitate consultation for the patient, in accordance with an example embodiment;

FIG. 7 is an example representation of another UI displayed to the telehealth provider to facilitate multiple input data fields for accessing and managing consultations assigned to the telehealth provider, in accordance with one embodiment of the present disclosure;

FIG. 8 is an example representation of a UI displayed to the telehealth provider for generating and managing billing details for each consultation, in accordance with one embodiment of the present disclosure;

FIG. 9 is an example representation of another UI displayed to the telehealth provider for accessing and managing assigned consultations or unassigned consultation to the telehealth provider, in accordance with one embodiment of the present disclosure;

FIG. 10 is an example representation of another UI displayed to the call center department for accessing and managing assigned consultations assigned to different telehealth providers, in accordance with one embodiment of the present disclosure;

FIG. 11 illustrates a flow diagram of another method for integrating multiple processes involved in telehealth consultation procedure, in accordance with one embodiment of the present disclosure; and

FIG. 12 illustrates a simplified block diagram of a user device, for example, a mobile phone or a desktop computer capable of implementing the various embodiments of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

The term “telehealth provider server system” used throughout the description refers to a server that executes and manages the complete process of telehealth consultation between the patient and the telehealth doctors across a distance. Further, the term “telehealth provider facility” used throughout the description refers to a facility which is connected to the telehealth provider server system and includes different departments associated to different processes involved in the process of telehealth consultation. Further, the term “telehealth provider” used throughout the description refers to a doctor who is registered with the telehealth provider server system for giving telehealth consultation to the patients. The term “medical facility”, used throughout the description, refers to a hospital who is registered with the telehealth provider server system for telehealth consultation.

### Overview

Various example embodiments of the present disclosure provide an integrated system and method for providing telehealth consultation. In an example embodiment, a consultation request to consult a doctor is received from a patient by a medical facility who has opted for telehealth facilities provided by a telehealth provider server system. Upon receiving the consultation request from the patient, the medical facility asks the patient few necessary details such as, but not limiting to, name of the patient, age of the patient, residential address of the patient, reason for consulting a doctor, and a type of visit such as emergency visit or a regular checkup visit. The medical facility sends the consultation request of the patient to the telehealth provider server system either by calling to a telehealth provider facility associated with the telehealth provider server system or by using an installed telehealth application portal hosted by the telehealth provider server system. A staff member at the call center of the telehealth provider facility attends the call from the medical facility and registers the consultation request of the patient along with the necessary details of the patient. Alternatively, the call center at the telehealth provider facility receives the consultation request from the medical facility via the telehealth application portal. The call center sends the consultation request along with the necessary details to the telehealth provider server system.

The telehealth provider server system checks the type of visit of the patient and process accordingly. In case the patient's visit is an emergency visit, the telehealth provider server system extracts a list of all the on-duty telehealth providers (interchangeably referred as “doctors”) along with their availability status and an estimated waiting time if they are busy with another consultation session. The availability status of each telehealth provider is continuously monitored and updated by the telehealth provider server system. The telehealth provider server system determines one telehealth provider from the multiple telehealth providers whose estimated waiting time is minimum among all the on-duty telehealth providers. The telehealth provider server system selects the telehealth provider with the least estimated waiting time and assigns the consultation request to the selected telehealth provider. The selected telehealth provider receives the assigned consultation request and accepts the consultation request. The telehealth provider server system sends a notification stating the consultation request is confirmed with the telehealth provider and the associated estimated waiting time to the telehealth provider facility. The telehealth provider facility sends the notification received from the telehealth provider server system to the medical facility.

In case the patient's visit is a regular health checkup visit, in most of the cases, the patient books an appointment with a primary doctor who is taking care of consultation of the patient in past visits of the patient. However, if the patient has not booked an appointment, then the telehealth provider server system will check who is the primary doctor if the patient is a registered patient, otherwise, the telehealth provider system assigns an earliest available doctor from the plurality of doctors. The availability status of the doctor can be for example, but not limited to “available now”, “not available”, “busy with other patient”, or “on leave”. Alternatively, or additionally, the doctor can share their live video with the telehealth provider server system using a camera module in a doctor application interface hosted by the telehealth provider server system. The telehealth provider server system monitors the live video of the doctor and determines the availability status of the doctor. The doctor may also send an estimated time required to finish an on-going session to the telehealth provider server system. The telehealth provider server system generates a message based on the availability status of the doctor and the estimated time provided by the doctor for the on-going session for example, “Your session is confirmed with Dr. K and it will start after 45 minutes as the doctor is busy in consulting another patient.” The telehealth provider server system sends the generated message to the call center. The staff at the call center shares the message to the medical facility.

Upon receiving the availability status of the doctor, the telehealth provider server system tracks the on-going consultation session of the assigned telehealth provider and determines whether the on-going consultation session is finished. When the on-going consultation session of the assigned telehealth provider is finished, the telehealth provider server system notifies the telehealth provider facility about the availability of the doctor and starting the session with the patient. The telehealth provider facility notifies the medical facility about the availability of the doctor and invites the patient for joining the session with the doctor.

The session for health consultation of the patient starts with the selected doctor. The doctor logins an application portal hosted by the telehealth provider server system using a login-id and password. The doctor accesses few features of the application portal for which an access is given to the doctor such as list of patients assigned to the doctor, details of each assigned patient, a consultation note option for creating medical notes during consultation of the patient, or a billing option for creating a billing form for the consultation. The doctor prepares the medical notes about his consultation of the patient in the application portal and submits the medical notes (interchangeably referred as “medical report”) to the telehealth provider server system after the session is finished. The doctor also fills the billing form including details such as the patient's name, the name of the medical facility, diagnostic codes, procedural codes, time taken for consultation etc. The doctor submits the bill form to the telehealth provider server system after the session is finished.

The telehealth provider server system generates a bill from the submitted bill form and optionally generates a medical report from the medical notes provided by the doctor. The telehealth provider server system sends the generated bill and the medical report to the telehealth provider facility. The telehealth provider facility sends the medical report and the bill to the patient based on a request from the patient. The medical report and the bill can be shared to the registered mobile number or the registered mail-id of the patient. The telehealth provider facility initiates a payment process for the consultation after the consultation for the amount mentioned in the bill. It shall be noted that the telehealth provider facility may initiate a payment process for the consultation even before the consultation by saving card details of the patient. The telehealth provider facility also sends a feedback form to the patient for providing feedback of the patient about the consultation given by the doctor and services provided by the telehealth provider facility.

The medical facility sends the feedback form filled by the patient to the telehealth provider facility. The telehealth provider facility sends the feedback form filled by the patient to the telehealth provider server system. The telehealth provider server system saves the feedback form in a database along with the respective consultation request of the patient. Further, the telehealth provider server system includes a listing of a group of experienced doctors for reviewing quality of consultation and treatment given by the telehealth providers. At least one experienced doctor (interchangeably referred to as “quality analyst”) reviews the medical notes submitted by the telehealth provider and reviews the consultation and diagnosis provided by the telehealth provider to the patient. At least one experienced doctor submits his assessment report about the consultation and diagnosis provided by the telehealth provider to the patient to the telehealth provider server system.

The telehealth provider server system further checks whether there is a billing gap based on a census record and accordingly notifies the billing and timesheet department of the telehealth provider facility. The telehealth provider server system ensures that the telehealth providers have a list of their assigned patients by the facility to make sure that each patient, requiring a visit at each location, receives timely consultation and the required documentation is completed in time.

Therefore, the telehealth provider server system provides a timely and convenient consultation to the patient as each doctor's availability is being monitored and the telehealth provider server system is able to commit nearly accurate estimation of waiting time to the patient and avoids the hassle of multiple discussions with the patient and doctor for connecting a session between them. Further, as the availability of the doctor is always updated there is no need to disturb the doctor during his consultation for assigning another consultation. Further, the documentation of the consultation and diagnosis of the patient medical condition performed by the telehealth provider is also managed by the telehealth provider server system in a timely manner. The documentation format given to the telehealth provider by the telehealth provider server system for preparing the medical notes of the consultation is a very detailed format so that no information gets missed out. Therefore, the documentation of the consultation and diagnosis of the patient medical condition which plays a very important role in the treatment of the patient going forward is very efficiently performed by the telehealth provider server system.

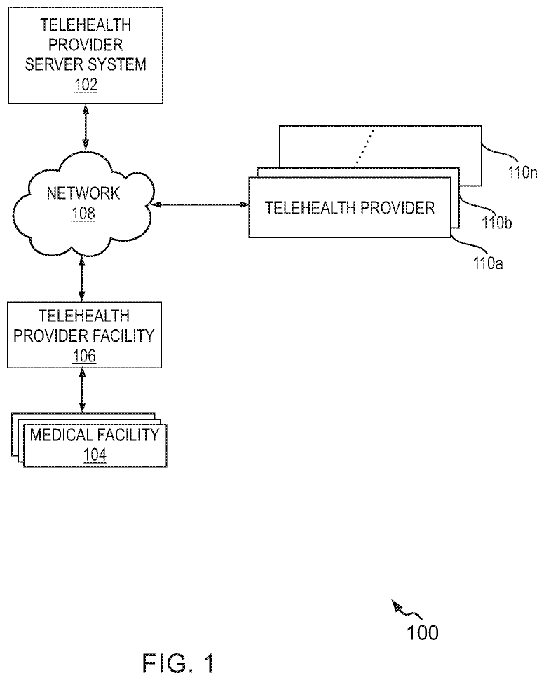

Various example embodiments of present invention are described hereinafter with reference to FIGS. 1 to 12. An environment for integrating multiple processes involved in telehealth consultation procedure using an integrated telehealth provider system and an integrated telehealth provider method is explained in detail with reference to FIG. 1.

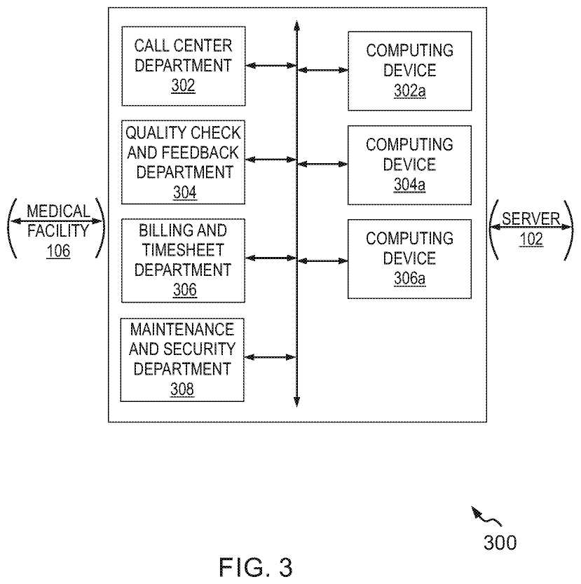

FIG. 1 illustrates an exemplary representation of an environment 100, in which at least some example embodiments of the present disclosure can be implemented. The environment 100 is exemplarily shown as a telehealth provider system 100 including an integrated telehealth management server system (also referred to as “a telehealth provider server system”) 102, a plurality of medical facilities 104 (also referred to herein as ‘hospitals 104’), a telehealth provider facility 106 comprising different departments each equipped with at least one computing device 302a (shown in FIG. 3) connected with the telehealth provider server system 102, a telehealth provider/doctor 110 associated with the telehealth provider server system 102 via a computing device (not shown in FIG. 1). Examples of the telehealth provider facility 106 may include any private medical establishments such as, hospital, clinic or government establishments such as, government hospital and clinic or any such place equipped with the application interface portal hosted by the telehealth provider server system 102 where medical facility 104 can connect for consultation for the patients visiting the medical facility 102. In various embodiments, the computing device 302a can be a mobile phone, laptop, personal digital assistant or a computer system operated by a staff of the department for connecting the patients and the telehealth providers/doctors for a health consultation. Examples of the computing device associated with the telehealth providers 110 include, but are not limited to, a personal computer (PC), a tablet device, a personal digital assistant (PDA), a smartphone and a laptop. It shall be noted that the term ‘telehealth provider facility’ may also refer to the online portal of the telehealth provider server system 102. The telehealth provider server system 102, the telehealth provider facility 106, the medical facilities 104 and the telehealth providers 110 communicate with each other via a network 108. It shall be noted that a single medical facility 104 is considered for further explanations for the sake of brevity and without limiting the scope of the plurality of medical facilities being associated with the telehealth provider server system 102. Similarly, a plurality of telehealth provider facilities 106 may be present at different locations and communicate with the telehealth provider server system 102 for the telehealth consultation.

In an example scenario, the medical facility 104 receives a consultation request from a patient. The medical facility 104 sends the consultation request of the patient to the telehealth provider facility 106 using a call, a message or an application portal hosted by the telehealth provider server system 102. The consultation request comprises, among other things, a name of the patient, a reason for consultation, type of visit, date of visit, number of visits, an account number of the patient or emergency contact member. The telehealth facility 106 includes a call center department 302 (shown in FIG. 3) which receives the consultation request of the patient from the medical facility 104. A staff member handling the call center department 302 of the telehealth provider facility 106 accesses a call center application interface (hereinafter referred as “Call API”) via a computing device associated with the staff member. The Call API is configured to communicate with the telehealth provider server system 102 and send instructions to the telehealth provider server system 102 to perform specific functions. The Call API can be a web-based application interface or a device-based application interface. The staff member opens the Call API into the computing device and logs in by using a login-id and password. The staff member sends the consultation request of the patient to the telehealth provider server system 102 via the Call API. The telehealth provider server system 102 comprises a plurality of applications for performing different functions.

The telehealth provider server system 102 receives the consultation request from the telehealth provider facility 106. Upon receiving the consultation request of the patient, the telehealth provider server system 102 determines the type of visit of the patient such as whether the visit is an emergency visit or a regular health checkup visit. The telehealth provider server system 102 selects one telehealth provider (e.g., 110a) from the plurality of telehealth providers 110a-110n based on the type of visit of the patient and assigns the consultation request to the selected telehealth provider 110a.

In an example embodiment, in case of an emergency visit or for a new patient, the telehealth provider server system 102 extracts a list of on-duty telehealth providers 110 along with information about each telehealth provider. The information comprises an availability status of each telehealth provider and an estimated waiting time required by each telehealth provider for starting the session for any new consultation request. The telehealth provider server system 102 renders the information to the telehealth provider facility 106 via the Call API. The staff member of the call center department 302 reads the information on the Call API and determines a telehealth provider 110a with least waiting time among all the telehealth providers in the list. The telehealth provider facility 106 sends a notification to the telehealth provider server system 102 for assigning the consultation request of the patient to the selected telehealth provider 110a.

In case of a regular health checkup visit, the telehealth provider server system 102 selects a telehealth provider 110b (hereinafter referred as “primary telehealth provider 110b”) among the on-duty telehealth providers. The primary telehealth provider 110b is the doctor who was previously assigned to the patient and has taken care of consultation of the patient from previous visits. The telehealth provider server system 102 also extracts information related to the primary telehealth provider 110b such as an availability status of the primary telehealth provider 110b along with an estimated waiting time required by the primary telehealth provider 110b for starting the session with the patient for the consultation request. The telehealth provider server system 102 renders the information to the telehealth provider facility 106 via the Call API. The staff member of the call center department 302 reads the information on the Call API and selects the primary telehealth provider 110b. The telehealth provider facility 106 sends a notification, via the Call API, to the telehealth provider server system 102 for assigning the consultation request of the patient to the selected telehealth provider 110b, which is the primary telehealth provider 110b.

The telehealth provider server system 102 sends the new consultation request to the selected telehealth provider (110a or 110b) selected based on the type of visit and whether the patient is new or already registered. The selected telehealth provider (110a or 110b) accepts the consultation request and sends the approval to the telehealth provider server system 102. The telehealth provider server system 102 notifies the telehealth provider facility 106 about the approval from the selected telehealth provider (110a or 110b). The telehealth provider facility 106 sends a notification message to the medical facility 104 stating that the selected telehealth provider (110a or 110b) is assigned. The notification message also includes an approximate waiting time required for starting the session of the patient with the selected telehealth provider (110a or 110b).

The telehealth provider server system 102 along with the telehealth provider facility 106 connects the patient with the selected telehealth provider (110a or 110b) once an on-going session of the selected telehealth provider (110a or 110b) gets over. It shall be noted that in case the patient is an old patient the telehealth provider server system 102 provides access to an electronic medical record (EMR) of the patient to the selected telehealth provider (110a or 110b) when requested by the selected telehealth provider (110a or 110b). The telehealth provider server system 102 creates the EMR for each patient using a clinical notes application and saves the created EMR for each patient in a database. The telehealth provider server system 102 also provides access to a tele-education (Tele-Ed) notes application to the selected telehealth provider (110a or 110b) for preparing medical notes during the consultation of the patient. It shall be noted that the telehealth providers 110 communicate with the telehealth provider server system 102 and the patient via a provider application interface installed on the computing device associated with the telehealth providers 110.

The telehealth provider server system 102 monitors the session of the consultation of the patient with the selected telehealth provider (110a or 110b) and receives a notification about completion of the session from the selected telehealth provider (110a or 110b). The telehealth provider server system 102 also generates a medical report and bill for the consultation of the patient based on medical notes provided by the selected telehealth provider (110a or 110b). The telehealth provider server system 102 sends the medical report and bill for the consultation of the patient to the telehealth provider facility 106. A billing and timesheet department 306 (shown in FIG. 3) of the telehealth provider facility 106 receives the bill via a billing application interface (hereinafter referred as “Billing API”) and reviews the bill and sends the bill to the medical facility 104. Further, the call center department 302 of the telehealth provider facility 106 receives the medical report via the Call API and sends the medical report to the patient. The telehealth provider server system 102 creates or updates the EMR of the patient based on the medical report. The telehealth provider server system 102 stores the updated EMR of each patient along with the bill of each visit in the database for future reference.

The telehealth provider facility 106 (specifically a quality check and feedback department 304, (see FIG. 3)) sends a feedback form to the patient for submitting feedback based on his/her experience during the consultation process with the selected telehealth provider (110a or 110b). The telehealth provider facility 106 sends the received feedback form to the telehealth provider server system 102 via a quality check application interface (QC API). The telehealth provider server system 102 stores the feedback form in the database along with the consultation request. The QC API is configured to provide communication between the quality check and feedback department 304 and the telehealth provider server system 102.

In at least one embodiment, the telehealth provider server system 102 further consults a group of experienced doctors which are part of the quality check and the feedback department 304 of the telehealth provider facility 106 for reviewing quality of consultation and treatment given by the telehealth providers. At least one experienced doctor (interchangeably referred as “quality analyst”) reviews the medical notes submitted by the selected telehealth provider (110a or 110b) and reviews the consultation and diagnosis provided by the selected telehealth provider (110a or 110b) to the patient. The at least one experienced doctor submits his assessment report about the consultation and diagnosis provided by the selected telehealth provider (110a or 110b) to the telehealth provider server system 102 by logging into the QC API hosted by the telehealth provider server system 102. The QC API is configured to provide communication between the quality analyst and the telehealth provider server system 102.

The billing and timesheet department 306 of the telehealth provider facility 106 manages, via the billing API, payment of bills for multiple consultation requests received from multiple facilities. The billing and timesheet department 306 of the telehealth provider facility 106 access details of the patient from the billing API which were updated when the consultation request was received from the medical facility 104. The billing and timesheet department 306 of the telehealth provider facility 106 initiates the payment process for billing once the consultation is over. The billing and timesheet department 306 further access, via the billing API, information related to working hours and salaries of each staff of the telehealth provider system 100 for example, staff members handling different departments such as call center department, the quality check and feedback department, the billing and timesheet department, and maintenance and security department, the telehealth providers 110, and the quality analysts. The billing and timesheet department 306 of the telehealth provider facility 106 credits the salary of each employee associated with the telehealth provider system 100 in a timely manner based on their working hours and salary structures.

The telehealth provider server system 102 further checks whether there is a billing gap based on a census record and accordingly notifies the billing and timesheet department 306 of the telehealth provider facility 106. The census record is defined as the number of consultations performed by the telehealth provider 110. Once a consultation is completed, the telehealth provider 110 is supposed to create a bill for the consultation. Sometimes, the telehealth provider 110 might forget to bill the consultation or might pre-bill the consultation. These errors create billing gap where the amount of bills received from the telehealth provider 110 are not equal to the amount of consultations performed by the telehealth provider 110. In order to maintain integrity as well as ensuring that the appropriate compensation is being made, the billing and timesheet department 306 is notified if such discrepancy is found. The telehealth provider server system 102 ensures that the telehealth providers 110 have a list of their assigned patients to make sure that each patient requiring a visit at each location receives timely consultation and the required documentation is completed in time.

It shall be noted that the call center department 302, the quality check and feedback department 304, the billing and timesheet department 306, the telehealth providers 110, and the quality analysts may communicate with each other via the network 108.

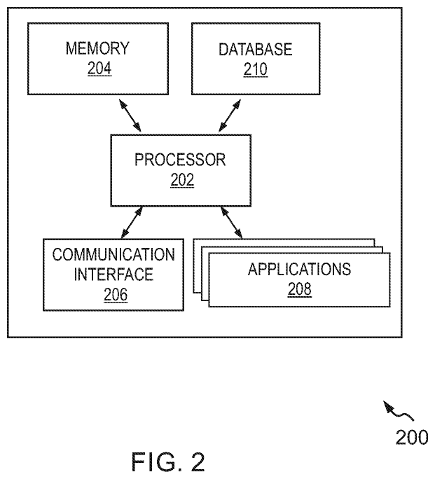

FIG. 2 illustrates a simplified block diagram of a server system 200 used for integrating multiple processes involved in telehealth consultation procedure, in accordance with one embodiment of the present disclosure. Examples of the server system 200 include, but are not limited to, the telehealth provider server system 102. The server system 200 includes a processor 202, a memory 204, a communication interface 206, a plurality of applications 208 and a database 210.

The processor 202 is configured for executing instructions. Instructions may be stored in, for example, but not limited to, the memory 204. The processor 202 may include one or more processing units (e.g., in a multi-core configuration).

The processor 202 is operatively coupled to the communication interface 206 such that the server system 200 is capable of communicating with a remote device such as the telehealth provider facility 106 and the telehealth providers 110. For example, the communication interface 206 may receive the consultation request from the telehealth provider facility 106, the approval message for the consultation request from the telehealth providers 110, medical notes from the telehealth providers 110 or billing forms from the telehealth providers 110. The communication interface 206 is further configured to send the consultation requests to respective telehealth providers 110, send the approval message to the telehealth provider facility 106, send the medical reports to the telehealth provider facility 106, and send the bills of each consultation request to the telehealth provider facility 106.

The processor 202 may also be operatively coupled to the database 210. The database 210 is any computer-operated hardware suitable for storing and/or retrieving data, such as, but not limited to, details of each patient received from the medical facility 104 along with an EMR of each patients consulted through the telehealth providers 110, medical notes submitted by the telehealth providers 110 for each consultation performed by the telehealth providers 110, a list of on-duty telehealth providers along with their availability status and estimated wait time for any new consultation, or billing forms and generated bills for each consultation. The database 210 may also store information related to a plurality of employee's accounts. Each employee account data includes at least one of an employee name, login credentials of the employee, an account number of the employee, salary structure of the employee, sign-in/sign-out time of each employee, and other details. The database 210 may include multiple storage units such as hard disks and/or solid-state disks in a redundant array of inexpensive disks (RAID) configuration. The database 210 may include a storage area network (SAN) and/or a network attached storage (NAS) system.

In some embodiments, the database 210 is external to the server system 200 and may be accessed by the server system 200 using a storage interface (not shown). The storage interface is any component capable of providing the processor 202 with access to the database 210. The storage interface may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 202 with access to the database 210.

The processor 202 is configured to enable each application from the plurality of applications 208 for performing different functions. The plurality of applications 208 comprises a time scheduler application (TMS App), a billing and timesheet application (Billing App), a quality check and feedback application (QC App), a tele-education notes application (Tele-Ed App), and a clinical notes application (Clinical notes App). The TMS App is configured to perform one or more functions such as: receiving the consultation requests from the telehealth provider facility 106; maintaining list of on-duty telehealth providers 110 along with their availability status and their estimated wait time for any new consultation; monitoring availability status of each on-duty telehealth provider along with their estimated wait time for any new consultation; identifying whether the patient is a new patient or an old patient; determining the type of visit of the patient such as an emergency visit or a regular health checkup; selecting an appropriate telehealth provider from the multiple on-duty telehealth providers; and facilitating consultation session between the patient and the telehealth provider.

The Billing App is configured to perform one or more functions such as: receiving a billing form filled by the selected telehealth provider (110a or 110b) for each consultation; generating a bill based on the billing form; tracking working hours of each employee based on sign-in/sign out details of each employee; crediting salary to each employee at the end of each month based on their respective salary structure; generating salary slips for each employee for each month of the year; and sending the generated bills and the salary slips to the database 210. The QC App is configured to perform one or more functions such as: providing access of the medical notes submitted by the telehealth providers 110 to the quality analysts along with the respective consultation request; receiving assessment report from the quality analysts regarding a quality of the consultation and diagnosis provided by the telehealth providers 110 for their respective consultations; and sending the assessment report to the database 210.

The Tele-Ed App is configured to provide a form in a predefined format to the telehealth providers during the consultation for making medical notes about the examination of a medical condition of the patient; sending the form including the medical notes to the clinical notes App. The clinical notes App is configured to prepare an EMR record for the patient based on the medical notes present in the form submitted by the telehealth providers 110.

Thereafter, the processor 202 is further configured to check whether there is a billing gap based on a census record and accordingly notifies the billing and timesheet department 306 of the telehealth provider facility 106. The processor 202 ensures that the telehealth providers 110 have a list of their assigned patients to make sure that each patient requiring a visit at each location receives timely consultation and the required documentation is completed in time.

In an example, the processor 202 may include one or more processors, microprocessors, data processors, co-processors, network processors, application specific integrated circuits (ASICs), controllers, programmable logic devices, chipsets, field programmable gate arrays (FPGAs), and/or some other component(s) that may interpret and/or execute instructions and/or data. The processor 202 may control the overall operation of the server system 200 based on an operating system and/or various applications.

FIG. 3 illustrates a work station example of a telehealth provider facility 300 including different departments, in accordance with an example embodiment. Examples of the telehealth provider facility 300 include, but are not limited to, the telehealth provider facility 106. The telehealth provider facility 300 includes call center department 302 associated with a computing device 302a, the quality check and feedback department 304 associated with a computing device 304a, the billing and timesheet department 306 associated with a computing device 306a, and maintenance and security department 308. All the departments may communicate with each other via the telehealth provider server system 102.

A staff member handling the call center department 302 of the telehealth provider facility 106 access a call center application interface (hereinafter referred to as “Call API”) via the computing device 302a. The Call API is configured to communicate with the telehealth provider server system 102 and send instructions to the telehealth provider server system 102 to perform specific functions. The Call API can be a web-based application interface or a device-based application interface. The staff member opens the Call API into the computing device and logs in by using a login-id and password. The staff member sends the consultation request of the patient to the telehealth provider server system 102 via the Call API.

The quality check and feedback department 304 sends a feedback form, via the QC API installed in the computing device 304a, to the patient for submitting feedback based on his/her experience during the consultation process with the selected telehealth provider (110a or 110b). The telehealth provider facility 106 sends the received feedback form to the telehealth provider server system 102 via the QC API.

The quality check and feedback department 304 further comprises a group of quality analysts for reviewing quality of consultation and treatment given by the telehealth providers. At least one quality analyst reviews the medical notes submitted by the selected telehealth provider and reviews the consultation and diagnosis provided by the selected telehealth provider to the patient. The at least one quality analyst submits his assessment report, via QC API installed at a computing device associated with the quality analyst, about the consultation and diagnosis provided by the selected telehealth provider to the telehealth provider server system 102. The QC API is configured to provide communication between the quality analyst and the telehealth provider server system 102.

The billing and timesheet department 306 of the telehealth provider facility 106 initiates, via the billing API installed in the computing device 306a, the payment process for billing once the consultation is over. The billing and timesheet department 306 of the telehealth provider facility 106 further access, via the billing API, information related to working hours and salaries of each staff of the telehealth provider system 100 for example, staff members handling different departments such as call center department 302, the quality check and feedback department 304, the billing and timesheet department 306, and maintenance and security department 308, the telehealth providers 110, and the quality analysts. The billing and timesheet department 306 of the telehealth provider facility 106 credits the salary of each employee associated with the telehealth provider system 100 in a timely manner based on their working hours and salary structures.

The maintenance and security department comprises staff members responsible for cleanliness of the telehealth provider facility 106, maintaining supply of daily needs for running the telehealth provider facility 106 such as water supply, electricity supply, network data connections, proper functioning of hardware, maintaining security within the premises of the telehealth provider facility 106.

It shall be noted that the above-mentioned different departments access their respective computing devices configured to perform functions via APIs (such as Call API, Billing API, and QC API) hosted by the telehealth provider server system 102. In an alternate embodiment of the present disclosure, the telehealth provider facility 106 can be an online portal hosted by the telehealth provider server system 102 and the different departments (such as the center department 302, the quality check and feedback department 304, the billing and timesheet department 306) can be dedicated programs configured to perform their respective operations.

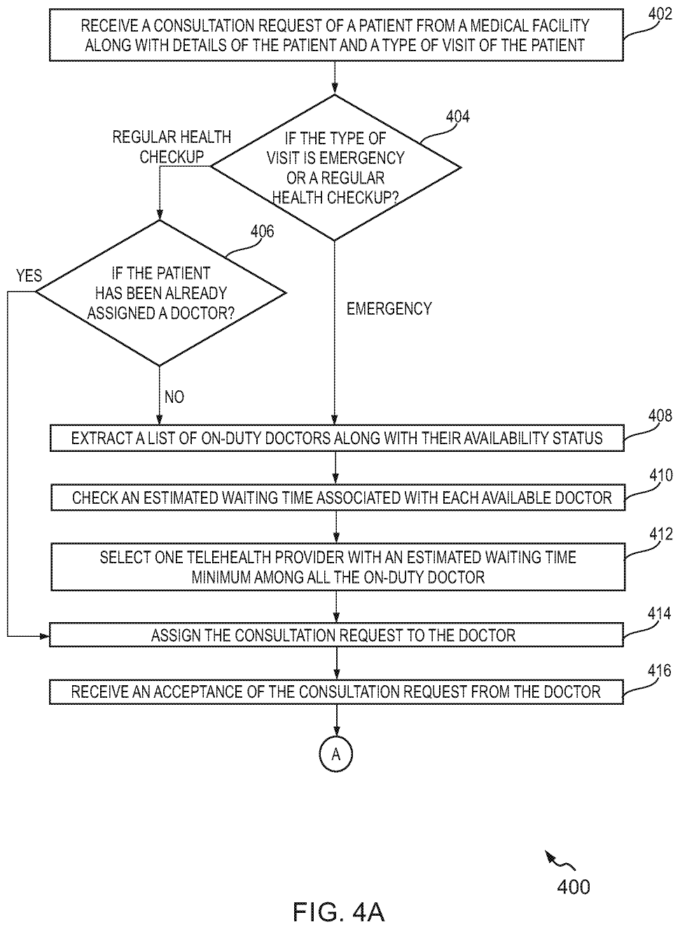

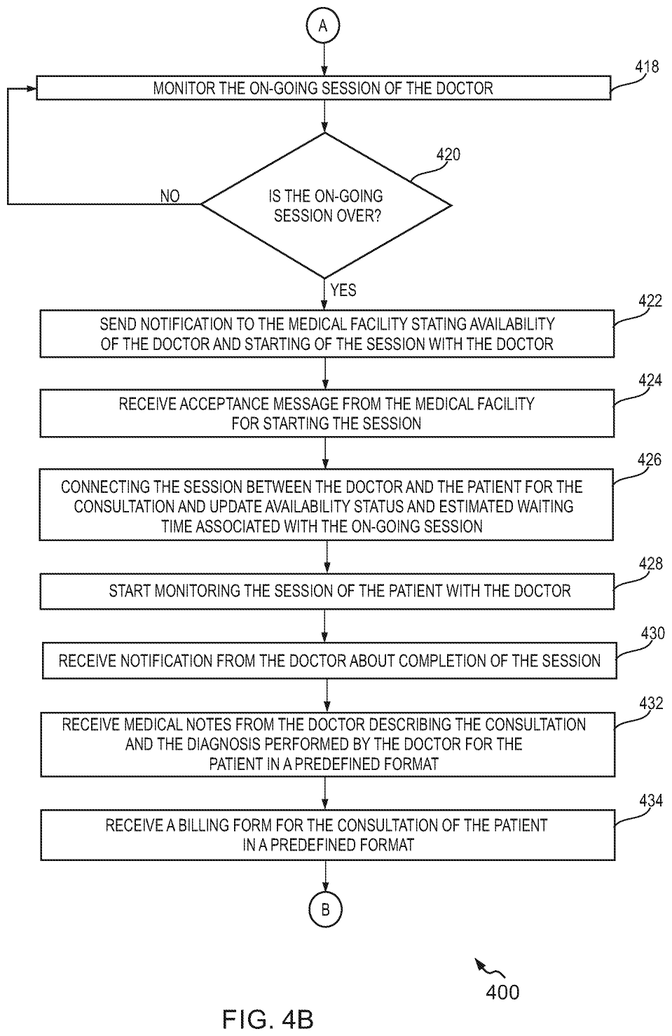

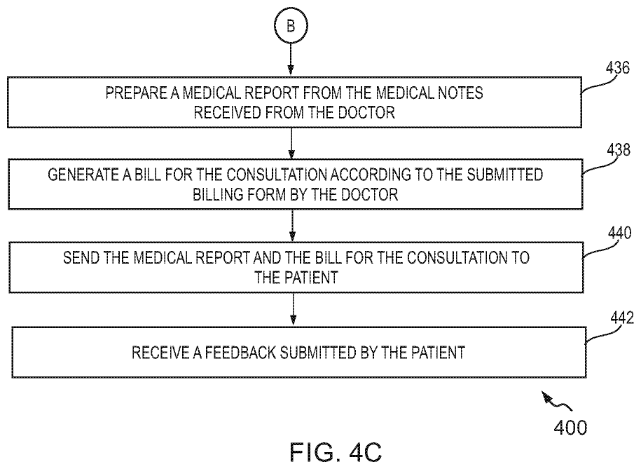

Referring now to FIG. 4, a flow diagram of a method 400 for integrating multiple processes involved in telehealth consultation procedure, is illustrated, in accordance with an example embodiment. The method 400 depicted in the flow diagram may be executed by a server system which may be the telehealth provider server system 102. Operations of the flow diagram and combinations of operation in the flow diagram, may be implemented by, for example, hardware, firmware, a processor, circuitry and/or a different device associated with the execution of software that includes one or more computer program instructions. The operations of the method 400 are described herein with the help of the telehealth provider server system 102, the telehealth provider facility 106 and the telehealth providers 110. The method 400 starts at operation 402 and ends at operation 442.

At operation 402, the method 400 includes receiving a consultation request of a patient from a medical facility 104 along with details of the patient and a type of visit of the patient. The details of the patient comprise the name of the patient, the reason for consultation, date of visit, number of visits, an account number of the patient or emergency contact member. The type of visit comprises one of an emergency visit or a regular health checkup visit.

At operation 404, the method 400 includes determining whether the type of visit is an emergency visit or a regular health checkup visit.

If the visit is a regular health checkup visit, the method 400 proceeds to operation 406 otherwise the method 400 proceeds to operation 408. At operation 408, the method 400 includes extracting a list of on-duty doctors along with their availability status. At operation 410, the method 400 includes checking an estimated waiting time associated with each available doctor. At operation 412, the method 400 includes selecting one telehealth provider with an estimated waiting time minimum among the on-duty doctors.

At operation 406, the method 500 includes determining whether the patient has already been assigned a doctor. For example, the patient may be an old patient with an assigned primary doctor or the patient may have booked an appointment prior to visiting the medical facility 104. In these cases, there will be an already assigned doctor for the patient.

If the patient has already been assigned with a doctor, the method 400 proceeds to operation 414, otherwise the method 400 performs operations 408-412 and then proceeds to operation 414. At operation 414, the method 400 includes assigning the consultation request to the doctor.

At operation 416, the method 400 includes receiving an acceptance of the consultation request from the doctor. At operation 418, the method 400 includes monitoring the on-going session of the doctor.

At operation 420, the method 400 includes determining whether the on-going session is over or not. If the session is over, the method 400 proceeds to operation 422 otherwise proceeds to operation 418. At operation 422, the method 400 includes sending a notification to the medical facility 104 stating the availability of the doctor and start of the session with the doctor.

At operation 424, the method 400 includes receiving an acceptance message from the medical facility 104 for starting the session. At operation 426, the method 400 includes connecting the session between the doctor and the patient for the consultation and update availability status and estimated waiting time associated with the on-going session.

At operation 428, the method 400 includes monitoring the session of the patient with the doctor. During the consultation, the doctor prepares medical notes related to the medical condition of the patient and treatment suggested by the doctor. The doctor may also share live video of the consultation session to the telehealth provider server system 102 for tracking the session. At operation 430, the method 400 includes receiving notification from the doctor about the completion of the session with the patient. Once the session is completed, the doctor submits the medical notes and a billing form respective to the consultation request to the telehealth provider server system 102.

At operation 432, the method 400 includes receiving medical notes from the doctor describing the consultation and the diagnosis performed by the doctor for the patient in a predefined format. At operation 434, the method 400 includes receiving a billing form for the consultation of the patient in a predefined format.

At operation 436, the method 400 includes preparing a medical report from the medical notes received from the doctor. At operation 438, the method 400 includes generating a bill for the consultation according to the submitted billing form by the doctor. At operation 440, the method 400 includes sending the medical report and the bill to the patient, and at operation 442, the method 400 includes receiving feedback submitted by the patient.

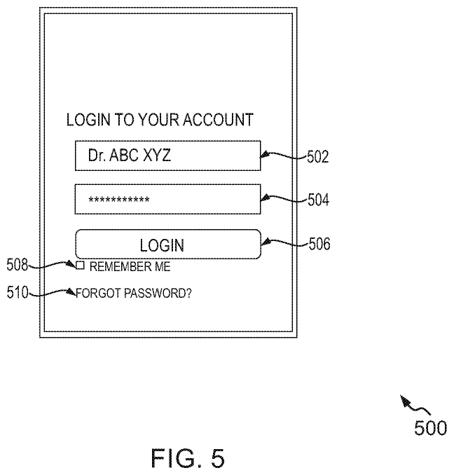

Referring now to FIG. 5, an example representation of a user interface (UI) 500 displayed to a display screen the computing devices of different employees or users of the telehealth provider system 100. The UI 500 is configured to render a login page for login to respective application interfaces (APIs), as illustrated in accordance with another example embodiment of the present disclosure.

The UI 500 rendered login page comprises input rows 502 and 504 for user-id and a password, respectively. The user/employee enters its user-id and password and clicks on the button 506 to login into a respective API on its respective computing device. For example, but not limited to, a staff member of the call center department 302 logins into Call API by using its assigned user-id and password. Similarly, a telehealth provider 110 logs into provider API on its computing device using its assigned login-id and password. The UI 500 includes one tick input box 508 for remembering the password for future logins and the UI 500 renders an active button 510 which on click redirects the user/employee to a page for re-entering a new password based on some secure authentication process.

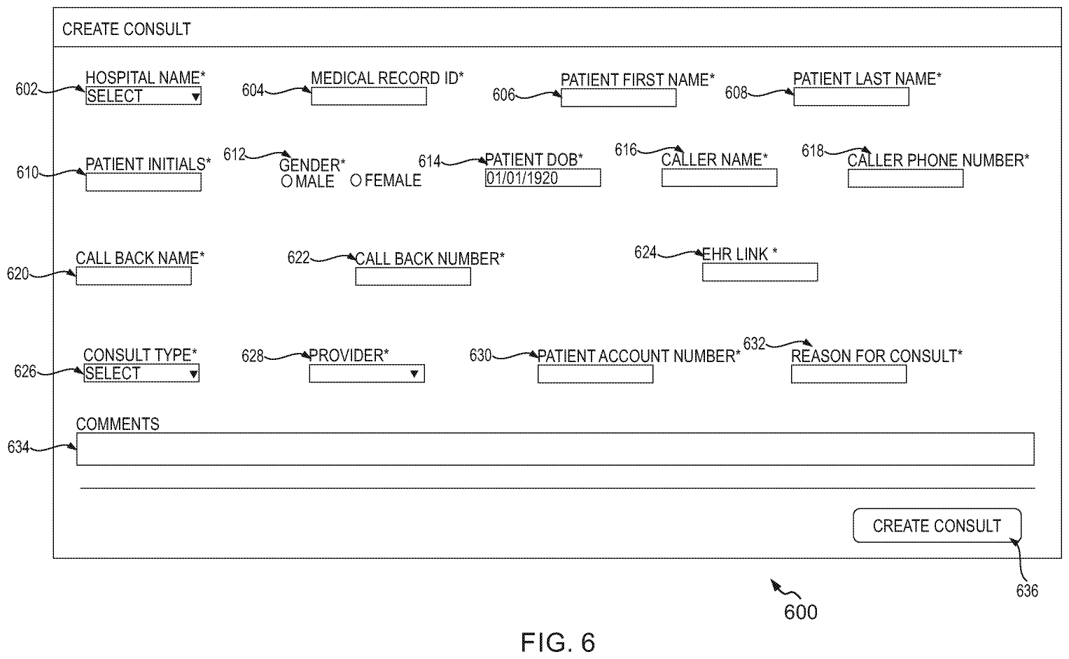

Referring now to FIG. 6, an example representation of a UI 600 is displayed to the staff member of the call center department 302 on a display screen of the computing device 302a. The UI 600 is configured to facilitate the creation of a consult for the patient corresponding to the received consultation request of the patient from the medical facility 104 after the staff member of the call center department 302 signs in the Call API with the login credentials.

The UI 600 renders multiple input data fields such as a hospital name 602 which is exemplarily shown as a dropdown input selection field, a medical record ID 604 used by the hospital, a patient's first name 606, a patient's last name 608, a patient's initials 610, a gender 612, a patient's date of birth (D.O.B.) 614, a caller name 616 i.e. the person who has sent the consultation request, a caller phone number 618, a call back name 620 i.e. the person who should be contacted once the consultation is completed, a call back number 622, an EHR link 624, a consult type 626, a provider 628 i.e. the assigned telehealth provider, a patient's account number 630, a reason for consult 632 and a comment section 634 for additional comments. It shall be noted that the input data fields in the UI 600 may be a drop-down input selection or a direct entry input data field or any other kind of input data field. Few of the input data fields are marked as (“*”) this indicates that these input data fields are mandatory to be filled.

The staff member of the call center department 302 fills respective data in the multiple input data fields and selects the create consult button 636 to create the consult of the patient for the consultation.

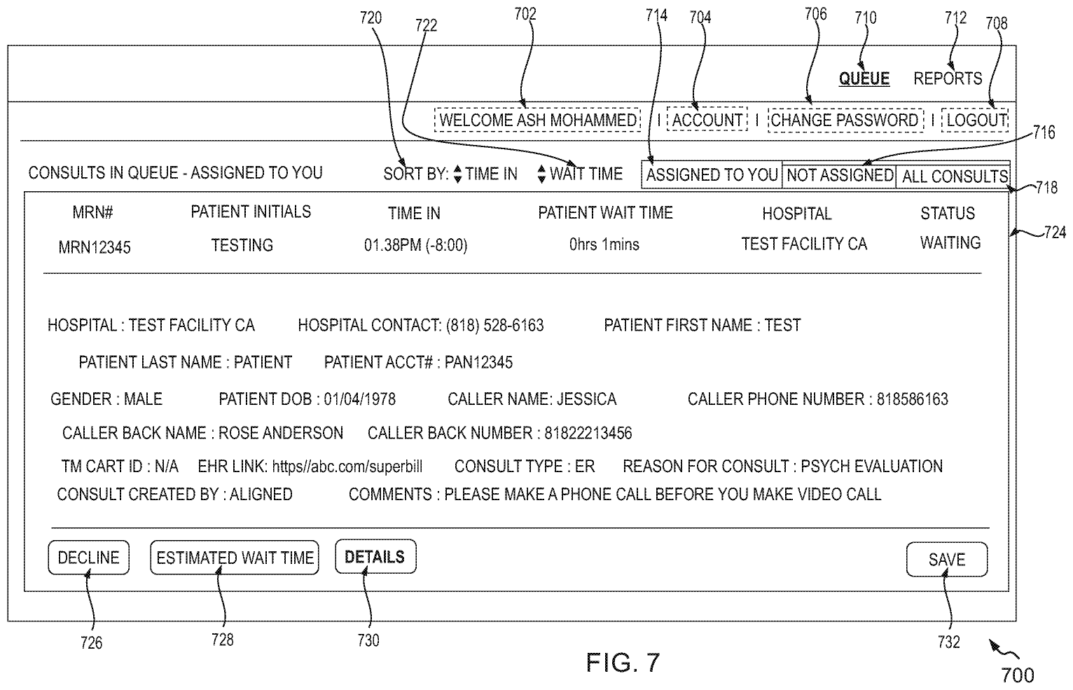

Referring now to FIG. 7, an example representation of a UI 700 is displayed to the telehealth provider 110 on a display screen of the computing device associated with the telehealth provider 110. The UI 700 is configured to facilitate multiple input data fields to the telehealth provider 110 for accessing and managing consultations assigned to the telehealth provider after the telehealth provider 110 signs in the provider API with the login credentials.

A shown in FIG. 7, the UI 700 renders multiple active access fields such as a telehealth provider profile field (shown as welcome ASH MOHAMMED) 702, an account field 704, a change password field 706, and a logout field 708. The UI 700 further renders active access fields such as Queue filed 710 providing access to details of the consultations which are assigned to the telehealth provider and are in queue, reports field 712 renders access to reports generated by the telehealth provider 110 or facilitates creating a new report for new consultation, an assigned to you field 714 showing consultations assigned to the telehealth provider 110, a not assigned filed 716 showing consults not assigned to the telehealth provider 110, an all consults filed 718 showing all the consultations received. The UI 700 further renders multiple fields such as sort by time field 720 or sort by wait time filed 722. The UI 700 further renders a box 724 including details of the consult, active selection input fields such as decline 726, estimated wait time 728 and details 730. The decline filed 726 is used to decline the consult, the estimated wait time field 728 is to enter an estimated wait time for the consult and details field 730 is to minimize or maximize the box 724. It shall be noted that the details provided in the box 724 are the details submitted by the staff member of the call center department 302 while creating the consult. The telehealth provider after accessing the consult and providing respective inputs selects the save 732 button to save the changes.

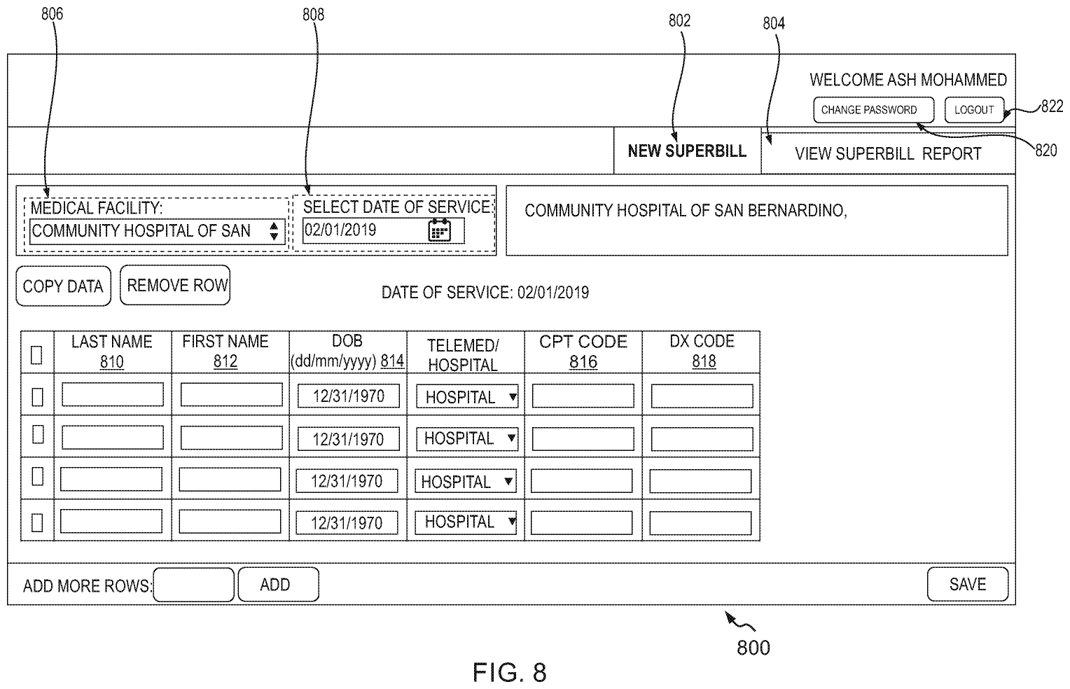

Referring now to FIG. 8, an example representation of a UI 800 is displayed to the telehealth provider 110 on the display screen of the computing device associated with the telehealth provider 110. The UI 800 is configured to facilitate multiple input data fields to the telehealth provider 110 for generating and managing billing details for each consultation of the patient.

A shown in FIG. 8, the UI 800 renders active access fields such as a new superbill field 802 and a view superbill report filed 804. The UI 800 further renders input data fields such as medical facility name 806, date of service 808 i.e. date on which the consultation was given, a last name filed 810 of the patient, a first name filed 812 of the patient, a D.O.B field 814, a CPT code field 816, and a DX code field 818. The telehealth provider 110 fills all the input fields and select save button to save the inputs. The UI 800 also renders fields such as, a change password field 820, and a logout field 822. The UI 800 is used for generating the billing form for each consultation taken by the telehealth provider 110.

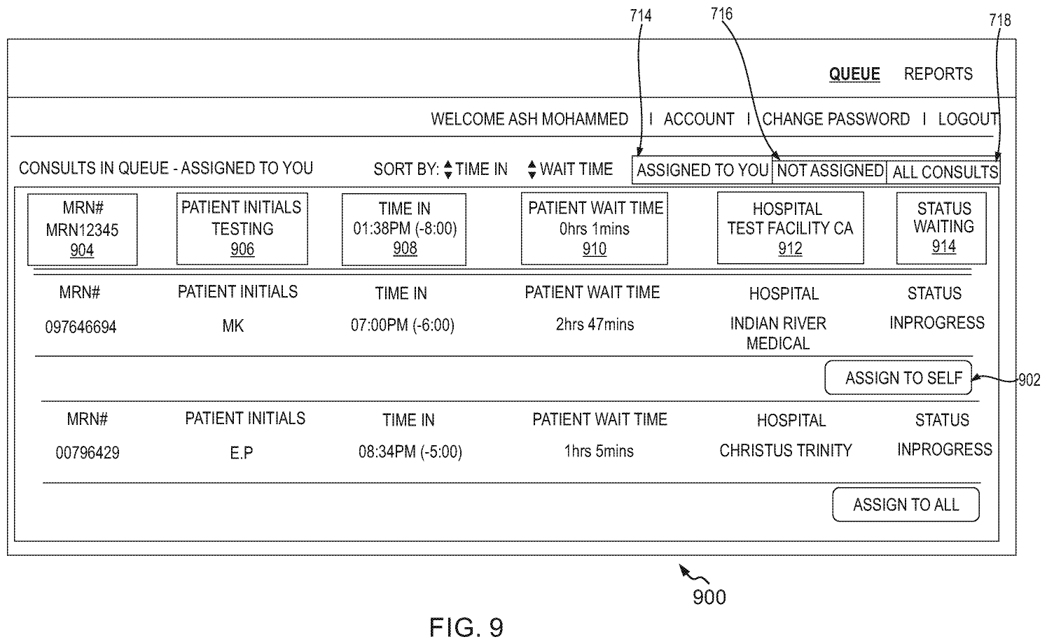

Referring now to FIG. 9, an example representation of a UI 900 is displayed to the telehealth provider 110 on the display screen of the computing device associated with the telehealth provider 110. The UI 900 is configured to facilitate multiple input data fields to the telehealth provider 110 for accessing and managing assigned consultations or unassigned consultation to the telehealth provider 110.

As shown in FIG. 9, the UI 900 renders multiple active access fields similar to those present in UI 700, for example, assign to you field 714, not assigned field 716 and the assign to all field 718, therefore the description of already mentioned fields is omitted for the sake of brevity. The UI 900 renders an assign to self-input field 902 for few consults present in the not assigned field 718. The telehealth provider 110 has the option of selecting the consult by clicking the assign to self-input field 902. The UI 900 also shows details of the consult such as MRN number 904 i.e. consultation number assigned to each consult, patients initials 906, a time-in of the patient 908, patient wait time 910, hospital 912 and status of the consult 914. It shall be noted that few fields such as sort by time, sort by wait time, have already explained in previous figures therefore their explanation is omitted for the sake of brevity.

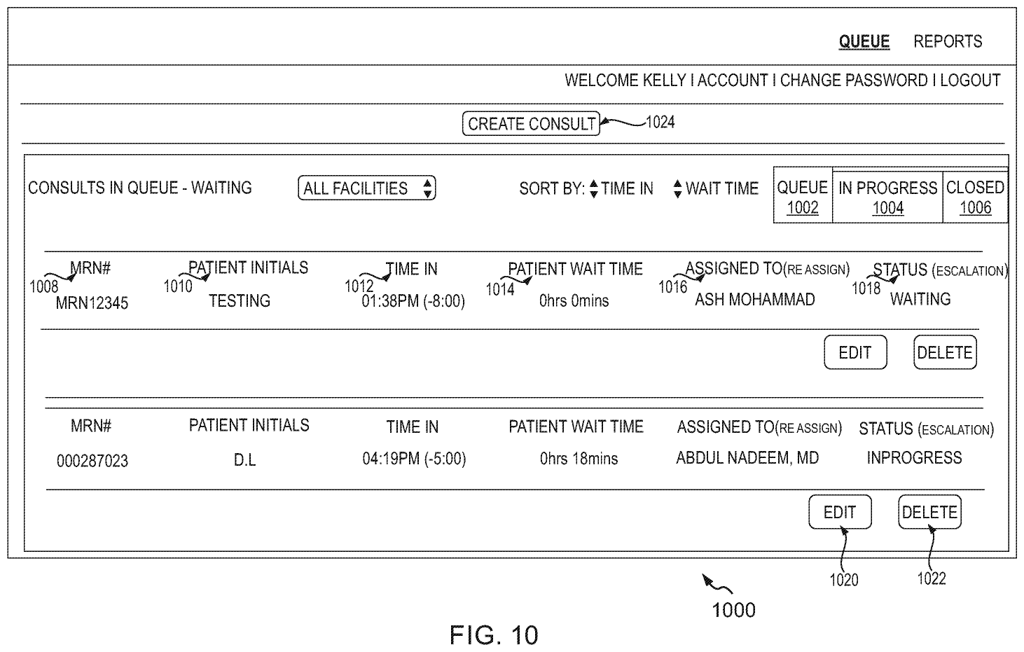

Referring now to FIG. 10, an example representation of a UI 1000 is displayed to the call center department 302 on the display screen of the computing device 302a. The UI 1000 is configured to facilitate multiple input data fields to the telehealth provider 110 for accessing and managing assigned consultations assigned to different telehealth providers.

As shown in FIG. 10, the UI 1000 renders multiple active access fields, for example, a “queue” field 1002, an “in process” field 1004, and a “closed” field 1006. The “queue” field 1002 when accessed by the staff member by clicking on the field, it shows a list of patients who are in a queue and waiting for their consultation with assigned telehealth provider. The “in process” field 1004 shows a list of patients whose consultation session is going on with the assigned telehealth provider. The “close” field 1006 shows the consultation sessions of the patients which are finished. The UI 1000 further shows details of each patient such as MRN number 1008, patient initials 1010, time in 1012, patient wait time 1014, assigned telehealth provider 1016, and status of the consultation request of the patient 1018. The UI 1000 also renders options such as Edit 1020 and delete 1022 on the display screen respective to each consult for providing an option of editing or deleting the consult. The staff member can select option 1024 “create consult” for creating new consult. It shall be noted that explanation of other fields in the UI 1000 which are explained in previous figures is omitted.

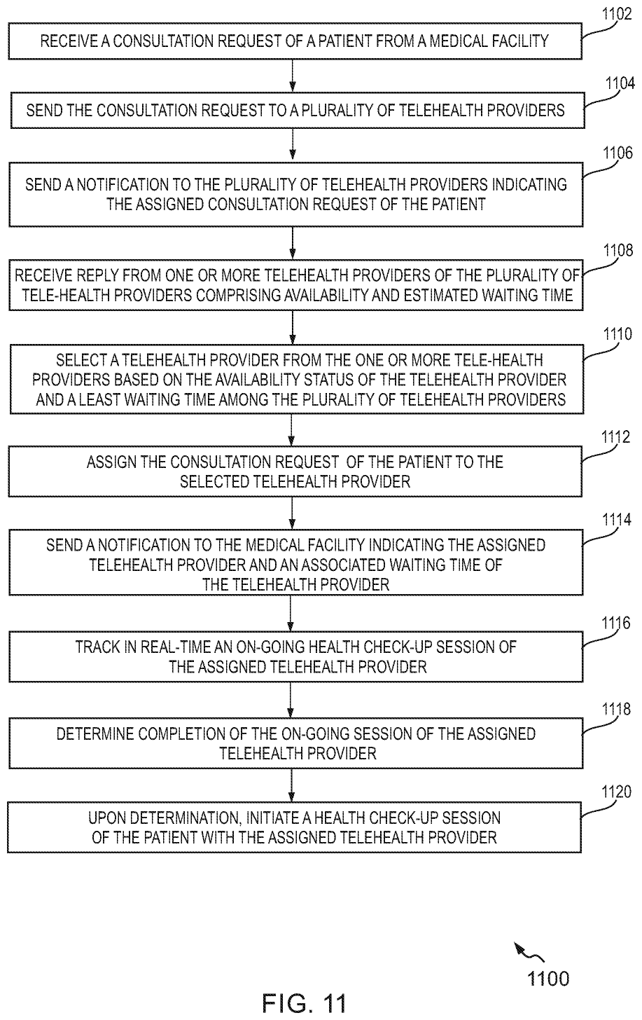

FIG. 11 illustrates a flow diagram of a method 1100 for integrating multiple processes involved in telehealth consultation procedure, in accordance with an example embodiment. The method 1100 depicted in the flow diagram may be executed by a server system which may be the telehealth provider server system 102. Operations of the flow diagram, and combinations of operation in the flow diagram may be implemented by, for example, hardware, firmware, a processor, circuitry and/or a different device associated with the execution of software that includes one or more computer program instructions. The operations of the method 1100 are described herein with the help of the telehealth provider server system 102, telehealth provider facility 106 and the telehealth providers 110. The method 1100 starts at operation 1102 and ends at operation 1120.

At operation 1102, the method 1100 includes receiving a consultation request of a patient from a medical facility 104 along with details of the patient and a type of visit of the patient. The details of the patient comprise the name of the patient, the reason for consultation, date of visit, number of visits, an account number of the patient or emergency contact member. The type of visit corresponds to an emergency.

At operation 1104, the method 1100 includes sending the consultation request for health consultation of the patient to a plurality of telehealth providers.

At operation 1106, the method 1100 includes sending a notification to the plurality of telehealth providers indicating the assigned consultation request.

At operation 1108, the method 1100 includes receiving a reply from one or more telehealth providers of the plurality of telehealth providers. The reply comprising an availability status of the one or more telehealth providers and a waiting time of the one or more telehealth providers required before starting a session for the health consultation of the patient.

At operation 1110, the method 1100 includes selecting a telehealth provider from the one or more telehealth providers based on the availability status of the telehealth provider and a least waiting time among the plurality of telehealth providers.

At operation 1112, the method 1100 includes assigning the consultation request to the selected telehealth provider. At operation 1114, the method 1100 includes sending a notification to the medical facility 104 indicating the assigned telehealth provider and an associated waiting time of the telehealth provider. At operation 1116, the method 1100 includes tracking in real-time an on-going health check-up session of the assigned telehealth provider. At operation 1118, the method 1100 includes determining completion of the on-going session of the assigned telehealth provider. At operation 1120, the method 1100 includes initiating a health check-up session of the patient with the assigned telehealth provider upon a determination that the on-going session is finished.

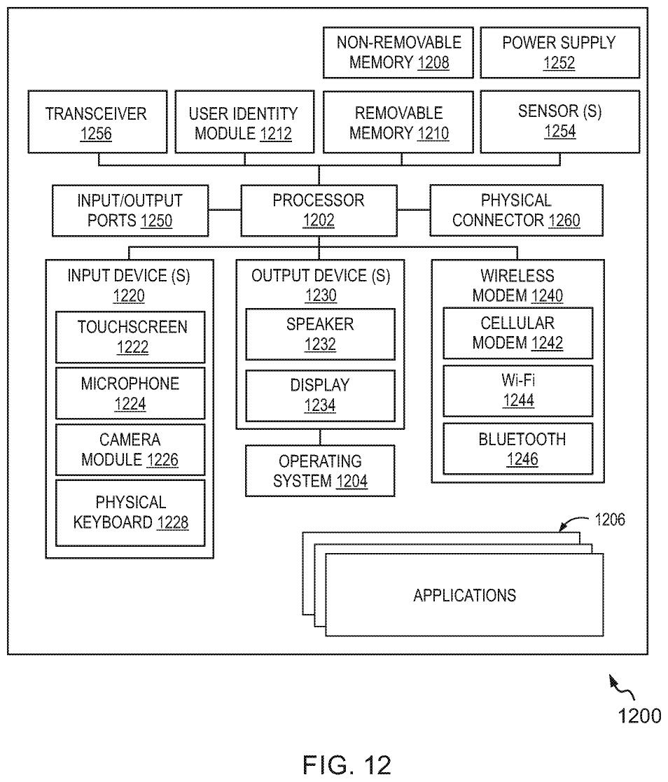

FIG. 12 shows a simplified block diagram of a user device 1200, for example, a mobile phone or a desktop computer capable of implementing the various embodiments of the present disclosure. For example, the user device 1200 may correspond to the computing devices 302a, 304a, 306a or computing device associated with telehealth providers 110 of FIG. 1 and FIG. 3. The user device 1100 is depicted to include one or more applications 1206 related to the present disclosure.

It should be understood that the user device 1200 as illustrated and hereinafter described is merely illustrative of one type of device and should not be taken to limit the scope of the embodiments. As such, it should be appreciated that at least some of the components described below in connection with that the user device 1200 may be optional and thus in an example embodiment may include more, less or different components than those described in connection with the example embodiment of FIG. 12. As such, among other examples, the user device 1200 could be any of an electronic device, for example, cellular phones, tablet computers, laptops, mobile computers, personal digital assistants (PDAs), mobile televisions, mobile digital assistants, or any combination of the aforementioned, and other types of communication or multimedia devices.

The illustrated user device 1200 includes a controller or a processor 1202 (e.g., a signal processor, microprocessor, ASIC, or other control and processing logic circuitry) for performing such tasks as signal coding, data processing, image processing, input/output processing, power control, and/or other functions. An operating system 1204 controls the allocation and usage of the components of the user device 1200 and support for one or more applications programs, that implements one or more of the innovative features described herein. The applications 1206 may include the TMS application, the billing and timesheet application, the quality check and feedback application, the tele-Ed notes application or the clinical notes application. Additionally, the applications 1206 may include common mobile computing applications (e.g., telephony applications, email applications, calendars, contact managers, web browsers, messaging applications such as USSD messaging or SMS messaging or SIM Tool Kit (STK) application) or any other computing application.

The illustrated user device 1200 includes one or more memory components, for example, a non-removable memory 1208 and/or a removable memory 1210. The non-removable memory 1208 and/or the removable memory 1210 may be collectively known as database in an embodiment. The non-removable memory 1208 can include RAM, ROM, flash memory, a hard disk, or other well-known memory storage technologies. The removable memory 1210 can include flash memory, smart cards, or a Subscriber Identity Module (SIM). The one or more memory components can be used for storing data and/or code for running the operating system 1204 and the applications 1206. The user device 1200 may further include a user identity module (UIM) 1212. The UIM 1212 may be a memory device having a processor built in. The UIM 1212 may include, for example, a subscriber identity module (SIM), a universal integrated circuit card (UICC), a universal subscriber identity module (USIM), a removable user identity module (R-UIM), or any other smart card. The UIM 1212 typically stores information elements related to a mobile subscriber. The UIM 1212 in form of the SIM card is well known in Global System for Mobile Communications (GSM) communication systems, Code Division Multiple Access (CDMA) systems, or with third-generation (3G) wireless communication protocols such as Universal Mobile Telecommunications System (UMTS), CDMA9000, wideband CDMA (WCDMA) and time division-synchronous CDMA (TD-SCDMA), or with fourth-generation (4G) wireless communication protocols such as LTE (Long-Term Evolution).

The user device 1200 can support one or more input devices 1220 and one or more output devices 1230. Examples of the input devices 1220 may include, but are not limited to, a touch screen/a display screen 1222 (e.g., capable of capturing finger tap inputs, finger gesture inputs, multi-finger tap inputs, multi-finger gesture inputs, or keystroke inputs from a virtual keyboard or keypad), a microphone 1224 (e.g., capable of capturing voice input), a camera module 1226 (e.g., capable of capturing still picture images and/or video images), and a keypad 1228. Examples of the output devices 1230 may include, but are not limited to a speaker 1232 and a display 1234. Other possible output devices can include piezoelectric or other haptic output devices. Some devices can serve more than one input/output function. For example, the touch screen 1222 and the display 1234 can be combined into a single input/output device.

A wireless modem 1240 can be coupled to one or more antennas (not shown in FIG. 12) and can support two-way communications between the processor 1202 and external devices, as is well understood in the art. The wireless modem 1240 is shown generically and can include, for example, a cellular modem 1242 for communicating at long range with the mobile communication network, a Wi-Fi compatible modem 1244 for communicating at short range with an external Bluetooth-equipped device or a local wireless data network or router, and/or a Bluetooth-compatible modem 1246. The wireless modem 1240 is typically configured for communication with one or more cellular networks, such as a GSM network for data and voice communications within a single cellular network, between cellular networks, or between the user device 1200 and a public switched telephone network (PSTN).

The user device 1200 can further include one or more input/output ports 1250 for establishing connection with peripheral devices including the POS terminal 1200, a power supply 1252, one or more sensors 1254 for example, an accelerometer, a gyroscope, a compass, or an infrared proximity sensor for detecting the orientation or motion of the user device 1200 and biometric sensors for scanning biometric identity of an authorized user, a transceiver 1256 (for wirelessly transmitting analog or digital signals) and/or a physical connector 1260, which can be a USB port, IEEE 1294 (FireWire) port, and/or RS-232 port. The illustrated components are not required or all-inclusive, as any of the components shown can be deleted and other components can be added.

Without in any way limiting the scope, interpretation, or application of the claims appearing below, a technical effect of one or more of the example embodiments disclosed herein is to provide methods and systems for integrated methods and systems for telehealth consultation providing following advantages and improvement over conventional telehealth consultation systems and methods:
Integrated and automated billing processing and on-time service delivery to the patients
No interruption to telehealth providers during the patient consultations as the status of the telehealth provider is constantly monitored without any interference in the consultation process.
Better communication among hospitals, call centre and doctors
Reduction in patients' wait time and faster documentation time
Instant generation of bills with no manual entry required
Automation of billing and census comparison
Faster documentation of patient notes and medical history
Efficiency in Telehealth Provider's documentation process
Help planning the facility visit in advance
Improved compliance with electronic notes and signatures
Real-time monitoring and reporting of consults wait-time, response time, admissions, and discharge of patients
Ability to capture telehealth provider's performance based on assessment of his treatment and diagnosis by experienced doctors.

The disclosed methods with reference to FIGS. 1 to 12, or one or more operations of the flow diagram 400 and 1100 may be implemented using software including computer-executable instructions stored on one or more computer-readable media (e.g., non-transitory computer-readable media, such as one or more optical media discs, volatile memory components (e.g., DRAM or SRAM), or nonvolatile memory or storage components (e.g., hard drives or solid-state nonvolatile memory components, such as Flash memory components) and executed on a computer (e.g., any suitable computer, such as a laptop computer, net book, Web book, tablet computing device, smart phone, or other mobile computing device). Such software may be executed, for example, on a single local computer or in a network environment (e.g., via the Internet, a wide-area network, a local-area network, a remote web-based server, a client-server network (such as a cloud computing network), or other such network) using one or more network computers. Additionally, any of the intermediate or final data created and used during implementation of the disclosed methods or systems may also be stored on one or more computer-readable media (e.g., non-transitory computer-readable media) and are considered to be within the scope of the disclosed technology. Furthermore, any of the software-based embodiments may be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

Although the disclosure has been described with reference to specific exemplary embodiments, it is noted that various modifications and changes may be made to these embodiments without departing from the broad spirit and scope of the disclosure. For example, the various operations, blocks, etc. described herein may be enabled and operated using hardware circuitry (for example, complementary metal oxide semiconductor (CMOS) based logic circuitry), firmware, software and/or any combination of hardware, firmware, and/or software (for example, embodied in a machine-readable medium). For example, the apparatuses and methods may be embodied using transistors, logic gates, and electrical circuits (for example, application specific integrated circuit (ASIC) circuitry and/or in Digital Signal Processor (DSP) circuitry).

Particularly, the server system 200 (e.g., the telehealth provider server system 102) and its various components such as the processor 205 and the database 210 may be enabled using software and/or using transistors, logic gates, and electrical circuits (for example, integrated circuit circuitry such as ASIC circuitry). Various embodiments of the disclosure may include one or more computer programs stored or otherwise embodied on a computer-readable medium, wherein the computer programs are configured to cause a processor or computer to perform one or more operations. A computer-readable medium storing, embodying, or encoded with a computer program, or similar language, may be embodied as a tangible data storage device storing one or more software programs that are configured to cause a processor or computer to perform one or more operations. Such operations may be, for example, any of the steps or operations described herein. In some embodiments, the computer programs may be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), DVD (Digital Versatile Disc), BD (BLU-RAY® Disc), and semiconductor memories (such as mask ROM, PROM (programmable ROM), EPROM (erasable PROM), flash memory, RAM (random access memory), etc.). Additionally, a tangible data storage device may be embodied as one or more volatile memory devices, one or more non-volatile memory devices, and/or a combination of one or more volatile memory devices and non-volatile memory devices. In some embodiments, the computer programs may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

Various embodiments of the invention, as discussed above, may be practiced with steps and/or operations in a different order, and/or with hardware elements in configurations, which are different than those which, are disclosed. Therefore, although the invention has been described based upon these exemplary embodiments, it is noted that certain modifications, variations, and alternative constructions may be apparent and well within the spirit and scope of the invention.

Although various exemplary embodiments of the invention are described herein in a language specific to structural features and/or methodological acts, the subject matter defined in the appended claims is not necessarily limited to the specific features or acts described above. Rather, the specific features and acts described above are disclosed as exemplary forms of implementing the claims.

1. An integrated telehealth management method, the method comprising:
receiving, by a server and through at least one application interface, a consultation request for a health consultation of a patient from a computing device of a telehealth provider facility, wherein the consultation request corresponds to an emergency, the consultation request comprising personal and health details of the patient;
extracting, by the server, telehealth provider information related to a plurality of telehealth providers, the telehealth provider information indicating an availability status of each of the plurality of telehealth providers and a waiting time of each of the plurality of telehealth providers required before starting a session for the health consultation of the patient;
causing, by the server and using the at least one application interface, the telehealth provider information to render on a display of the computing device of the telehealth provider;
receiving, by the server and through the at least one application interface, a notification from the computing device of the telehealth provider facility indicating a selection of a telehealth provider of the plurality of telehealth providers based on the availability status and a least waiting time among the plurality of telehealth providers of the telehealth provider information;
assigning, by the server, the consultation request to the selected telehealth provider, including:
sending, by the server and using the at least one application interface, a notification to the computing device of the medical facility indicating the assigned telehealth provider and an associated waiting time of the telehealth provider;
tracking, by the server and in real-time, an on-going health check-up session of the assigned telehealth provider;
determining, by the server, completion of the on-going health check-up session of the assigned telehealth provider;
upon determining the completion, initiating, by the server and using the at least one application interface, a communications session between a device of the patient and a device of the assigned telehealth provider;
determining, by the server, completion of the communications session;
generating, by the server, post-communications session information representing a medical report and a bill for the communications session based on input received, by the server and through the at least one application interface, from the computing device of the telehealth provider; and
sending, by the server and using the at least one application interface, the post-communications session information to the computing device of the telehealth provider facility.

2. The method as claimed in claim 1,
upon completion of the health check-up session of the patient with the assigned telehealth provider, rendering a billing form on a computing device associated with the assigned telehealth provider;
receiving inputs related to billing of the health consultation of the patient, the inputs comprising at least one of a name of the medical facility, a name of the patient, a time duration of the health check-up session of the patient, a diagnostic code, a current procedural terminology code, and a number of sessions of the patient;
generating a bill based on the inputs received from the assigned telehealth provider on the billing form; and
sharing the generated bill to the patient.

3. The method as claimed in claim 1, wherein generating post-communications session information comprises:
upon determining completion of the communications session, rendering a report form on the computing device of the assigned telehealth provider through the at least one application interface;
receiving, through the at least one application interface, inputs related to the health check-up session of the patient from the assigned telehealth provider on the report form, the inputs comprising at least one of a time duration of the health check-up session of the patient, telehealth provider's medical diagnosis, or a treatment suggested by the assigned telehealth provider;
generating the medical report based on the inputs received from the assigned telehealth provider on the report form; and
sending at least a portion of the post-communications session information representing the generated medical report to the device of the patient.

4. The method as claimed in claim 3, further comprising:
storing the post-communications session information along with the respective consultation request in a database.

5. The method as claimed in claim 4, further comprising:
providing access of the stored report form to a quality analyst for reviewing a quality of treatment given by the assigned telehealth provider;
receiving feedback information representing feedback from the quality analyst on the quality of treatment given by the assigned telehealth provider; and
generating an assessment report of the quality of treatment given by the assigned telehealth provider based on the feedback received from the quality analyst.

6. The method as claimed in claim 1, further comprising:
maintaining a timesheet of working hours and remuneration of each staff associated with a telehealth provider system;
generating a salary for each staff of the telehealth provider system; and
facilitating credit of the salary of each staff of the telehealth provider system.

7. The method as claimed in claim 1, further comprising:
creating an electronic health record of the patient; and
storing the electronic health record of the patient in a database.

8. An integrated telehealth management server system, the system comprising:
a memory configured to store instructions; and
a processor configured to execute the instructions stored in the memory and thereby cause the system to perform:
receive a consultation request for a health consultation of a patient from a medical facility, wherein the consultation request correspond to an emergency, the consultation request comprising personal and health details of the patient;
send the consultation request for health consultation of the patient to a plurality of telehealth providers;
send a notification to the plurality of telehealth providers indicating the consultation request;
receive a reply from one or more telehealth providers of the plurality of telehealth providers, the reply comprising an availability status of each of the one or more telehealth providers and a waiting time of each of the one or more telehealth providers required before starting a session for the health consultation of the patient;
select a telehealth provider from the one or more telehealth providers based on the availability status of the telehealth provider and a least waiting time among the plurality of telehealth providers;
assign the consultation request to the selected telehealth provider;
send a notification to the medical facility indicating the assigned telehealth provider and an associated waiting time of the telehealth provider;
track in real-time an on-going health check-up session of the assigned telehealth provider;
determine completion of the on-going health check-up session of the assigned telehealth provider; and
upon determination of the completion, initiate a health check-up session of the patient with the assigned telehealth provider.

9. The system as claimed in claim 8, wherein the processor is further configured to cause the system to perform:
upon completion of the health check-up session of the patient with the assigned telehealth provider, render a billing form on a computing device associated with the assigned telehealth provider;
receive inputs related to billing of the health consultation of the patient, the inputs comprising at least one of a name of the medical facility, a name of the patient, a time duration of the health check-up session of the patient, a diagnostic code, a current procedural terminology code, and a number of sessions of the patient;
generate a bill based on the inputs received from the assigned telehealth provider on the billing form;
share the generated bill to the patient; and
store the bill along with the respective consultation request in a database.

10. The system as claimed in claim 8, wherein the processor is further configured to cause the system to perform:
upon completion of the health check-up session of the patient with the assigned telehealth provider, render a report form on a computing device associated with the assigned telehealth provider;
receive inputs related to the health check-up session of the patient from the assigned telehealth provider on the report form, the inputs comprising at least one of a name of the medical facility, a name of the patient, a time duration of the health check-up session of the patient, telehealth provider's medical diagnosis, or a treatment suggested by the assigned telehealth provider;
generate a medical report based on the inputs received from the assigned telehealth provider on the report form;
share the generated medical report to the medical facility; and
store the medical report along with the respective consultation request in a database.

11. The system as claimed in claim 10, wherein the processor is further configured to cause the system to perform:
provide access of the stored report form to a quality analyst for reviewing a quality of treatment given by the assigned telehealth provider;
receive a feedback from the quality analyst on the quality of treatment given by the assigned telehealth provider; and
generate an assessment report of the quality of treatment given by the assigned telehealth provider based on the feedback received from the quality analyst.

12. The system as claimed in claim 8, wherein the processor is further configured to cause the system to perform:
maintain a timesheet of working hours and remuneration of each staff associated with a telehealth provider system;
generate a salary for each staff of the telehealth provider system; and
facilitate credit of the salary of each staff of the telehealth provider system.

13. The system as claimed in claim 8, wherein the processor is further configured to cause the system to perform:
create an electronic health record of each patient; and
store the electronic health record of each patient in a database.

14. An integrated telehealth management method, the method comprising:
receiving a consultation request for a health consultation of a patient from a medical facility, wherein the consultation request comprising personal and health details of the patient, and a type of the consultation request, the type of the consultation request is one of an emergency or a regular health-checkup;
determining whether the type of the consultation request is the emergency or the regular health-checkup;
upon determination that the type of the consultation request is the regular health-checkup, performing
assigning the consultation request for health consultation of the patient to a primary telehealth provider,
sending a notification to the primary telehealth provider indicating the assigned consultation request for health consultation of the patient,
receiving a reply from the primary telehealth provider, the reply comprises one of a video call, a phone call or a message from the primary telehealth provider, the reply indicating at least one of an availability status of the primary telehealth provider or a waiting time required before starting a session for the consultation request,
assigning the consultation request to the primary telehealth provider,
sending a notification to the medical facility indicating the associated waiting time for the primary telehealth provider,
tracking in real-time an on-going health check-up session of the primary telehealth provider,
determining completion of the on-going health check-up session of the primary telehealth provider, and
upon determining the completion, initiating a health check-up session of the patient with the primary telehealth provider; and
upon determination that the type of the consultation request is the emergency, performing
sending the consultation request for the health consultation of the patient to a plurality of telehealth providers,
sending a notification to the plurality of telehealth providers indicating the consultation request,
receiving a reply from one or more telehealth providers of the plurality of telehealth providers, the reply comprising an availability status of each of the one or more telehealth providers and a waiting time of each of the one or more telehealth providers required before starting a session for the health consultation of the patient,
selecting a telehealth provider from the one or more telehealth providers based on the availability status of the telehealth provider and a least waiting time among the plurality of telehealth providers,
assigning the consultation request for health consultation of the patient to the selected telehealth provider,
sending a notification to the medical facility indicating the associated waiting time for the selected telehealth provider,
tracking in real-time an on-going health check-up session of the selected telehealth provider,
determining completion of the on-going health check-up session of the selected telehealth provider, and
upon determining the completion, initiating a health check-up session of the patient with the selected telehealth provider.

15. The method as claimed in claim 14, further comprising:
upon completion of the health check-up session of the patient with one of the primary telehealth provider or the selected telehealth provider, rendering a report form on a computing device associated with one of the primary telehealth provider or the selected telehealth provider;
receiving inputs related to the health check-up session of the patient from one of the primary telehealth provider or the selected telehealth provider on the report form, the inputs comprising at least one of a name of the medical facility, a name of the patient, a time duration of the health check-up session of the patient, telehealth provider's medical diagnosis, or a treatment suggested by the assigned telehealth provider;
generating a medical report based on the inputs received from one of the primary telehealth provider or the selected telehealth provider on the report form; and
sharing the medical report to the patient.

16. The method as claimed in claim 15, further comprising:
storing the report form submitted by the primary telehealth provider along with the respective consultation request for the health consultation of the patient.

17. The method as claimed in claim 15, further comprising:
providing access of the stored medical report to a quality analyst for reviewing a quality of treatment given by one of the primary telehealth provider or the selected telehealth provider;
receiving a feedback from the quality analyst on the quality of treatment given by one of the primary telehealth provider or the selected telehealth provider; and
generating an assessment report of the quality of treatment given by one of the primary telehealth provider or the selected telehealth provider based on the feedback received from the quality analyst.

18. The method as claimed in claim 14, further comprising:
upon completion of the health check-up session of the patient with one of the primary telehealth provider or the selected telehealth provider, rendering a billing form on a computing device associated with one of the primary telehealth provider or the selected telehealth provider;
receiving inputs related to the health check-up session of the patient from one of the primary telehealth provider or the selected telehealth provider on the report form, the inputs comprising at least one of a name of the medical facility, a name of the patient, a time duration of the health check-up session of the patient, a diagnostic code, a current procedural terminology code, and a number of sessions of the patient;
generating a bill based on the inputs received from one of the primary telehealth provider or the selected telehealth provider on the billing form; and
sharing the generated bill to the medical facility.

19. The method as claimed in claim 14, further comprising:
maintaining a timesheet of working hours and remuneration of each staff associated with telehealth provider system;
generating salary for each staff of the telehealth provider system; and
crediting the salary of each staff of the telehealth provider system.

20. The method as claimed in claim 14, further comprising:
creating an electronic health record of each patient; and
storing the electronic health record of each patient in a database.
